(19) United States
(12) Patent Application Publication
(10) Pub. No.: US 2020/0323989 A1
(43) Pub. Date: Oct. 15, 2020

(54) BIO-SIMILAR EMU OIL COMPOSITION

(71) Applicants: Jeffrey Lokken, Buffalo, NY (US); Katherine Cwiklinski, Buffalo, NY (US)

(72) Inventors: Jeffrey Lokken, Buffalo, NY (US); Katherine Cwiklinski, Buffalo, NY (US)

(21) Appl. No.: 16/381,329

(22) Filed: Apr. 11, 2019

(51) Int. Cl. A61K 47/44; A61K 31/201; A61K 9/00

(52) U.S. Cl. CPC A61K 47/44; A61Q 19/00; A61K 9/0014; A61K 31/201

(57) ABSTRACT

A bio-similar emu oil composition. The oil composition is effective for treating a broad array of medical conditions including: inflammation; allergies; headaches; skin condition and imperfections; hair loss; muscle pain; and other maladies.

### FIELD OF INVENTION

This invention is directed to a bio-similar emu oil composition. The oil composition is effective for treating a broad array of medical conditions including: inflammation; allergies; headaches; skin conditions and imperfections; hair loss; muscle pain; and other maladies. Emu oil is also used as a popular cosmetic and personal care ingredient. It is particularly effective for emolliency, moisturizing, and skin penetration.

### BACKGROUND OF INVENTION

Many people suffer from a broad variety of medical problems and conditions that require some sort of medical treatment. For many of the medical conditions, an oil is the recommended treatment or may be an effective home treatment.

Because there are so many oils on the market, it would be beneficial to find a consistent oil composition that treats a broad array of medical conditions. The oil composition may be a component in a topical cream or lotion. Alternatively, the oil composition may be used for transdermal delivery.

One broad spectrum oil that is already available is emu oil. Emu (Dromaius novaehollandiae) are large birds that are indigenous to Australia. They are the second member of the large flightless birds and can reach 5 feet tall and 140 pounds. Emu are farmed around the world for their meat, leather, eggs, and oil. Australian Aboriginals used emu fat as a sun screen and incorporated emu meat in their diet. Early European settlers noted that the Aboriginals also used the fat to treat wounds and other skin conditions and to alleviate joint pain.

More recently, emu oil has been used in many personal care products and cosmetics. While the mechanism of action of emu oil has not been identified, it is thought to be beneficial because emu oil has a fatty acid composition close to that of humans. Many benefits of using emu oil have been identified. One such use is that of a topical skin penetrant that can be used in conjunction with other active ingredients to treat such ailments as acne, wrinkles, and alopecia (U.S. Pat. No. 9,757,324). Emu oil has been used in topical, therapeutic compositions that treat pain, inflammation, and swelling (U.S. Pat. No. 9,579,357). Emu oil can be used as a formulation base for the treatment of damaged skin by inhibiting microbial and fungal activity (U.S. Pat. No. 7,476,379). U.S. Pat. No. 9,987,248, demonstrates how emu oil is used as a transdermal delivery agent for various cannabinoids. US 2004/0185115 A1 discloses emu oil in therapeutic compositions that are adapted for cleansing and treating skin. As can be seen, emu oil is a broad-spectrum oil having many uses for medical conditions.

Importantly, emu oil has been tested for its anti-inflammatory and anti-arthritic properties in animals (Study To Determine If Emu Oil Showed Anti-Inflammatory, Anti-Arthritic Activity In Laboratory Animals by Dr. Peter Ghosh, Royal North Shore Hospital, Sydney, Australia Dr. Michael Whitehouse, University of Adelaide, Australia 1988). Emu oil is used for cosmetic purposes as well for such things as mineral oil replacement, as an oil with low comedogenicity, and as a non-irritating oil replacement (Moisturizing and Cosmetic Properties Of Emu Oil, A Double Blind Study Presented at the AEA national convention in Nashville, Tenn. August 1994 Alexander Zemtsov, M.D., M.S. Indiana University School of Medicine Monica Gaddis, Ph.D. Ball Memorial Hospital Victor Montalvo-Lugo, M.S. Ball Memorial Hospital). Many other therapeutic uses are described in U.S. Pat. No. 5,472,713 including prevention of stretch marks during pregnancy, treating cold and flu symptoms, lowering cholesterol, and treating and preventing headaches.

Due to the similarity of the fatty acid composition to that of human skin, emu oil is a desired cosmetic and skin care ingredient, and is often used as an emollient which has the property of softening and soothing the skin in addition to forming a barrier to help maintain skin hydration.

Overall, emu oil is a highly effective, broad spectrum oil for use in a wide variety of medical conditions. Unfortunately, emus must be slaughtered in order to obtain their fats (oils). Of particular concern, many people believe that the driving force behind the farming of emus is due to the collection of oil used for cosmetic purposes, rather than for the emu meat. With a growing vegan and vegetarian community and an increase in general attitudes towards cruelty-free products, it would be desirable to have a non-animal source of a bio-similar or bio-equivalent emu oil.

It should also be noted that emu oil is very expensive. Vegan alternatives are much more affordable, thereby enabling the benefits to be obtained by a larger percentage of the population.

### SUMMARY OF THE INVENTION

Accordingly, it is the subject of this invention to provide a non-animal bio-similar emu oil composition.

It is noted that the composition of the fatty acid isolates from emu oil are different depending on a variety of factors. That is, there isn't one fatty acid profile of emu oil, rather there are many different fatty acid profiles depending on several different factors, such as the sex of the emu, the body region where the oil was harvested from (front, back, neck, or internal, and the diet of the particular emu (Isolation and Characterization of Oil from Fatty Tissues of Emu Birds Farmed in India, Shiva Shanker Kaki, Thumu Ravinder, B V S K Rao, P P Chakrabarti and R B N Prasad* Centre for Lipid Research, CSIR-Indian Institute of Chemical Technology, Hyderabad).

Nonetheless, all emu oil fatty acid profiles contain a broad spectrum of essential fatty acids including omega-3 (mainly linolenic acid), omega-6 (mainly linoleic acid), omega-7 (mainly palmitoleic acid), and omega-9 (mainly oleic acid). These omega essential fatty acids, in addition to stearic acid and palmitic acid, are the six major constituents in emu oil comprising approximately 96% or more of the total composition. However, there is no single plant-based bio-equivalent or bio-similar oil. Specifically, there is no direct plant-based offset that consistently contains these main ingredients in the same or very similar ratios as to that of emu oil.

Thus, there is a need to create a consistent non-animal oil composition that provides similar benefits to that of emu oil. The present disclosure has identified a combination of the most important constituents of emu oil. Thus, the present disclosure provides a consistent non-animal oil composition that has similar properties and characteristics to emu oil.

In particular, the present disclosure has identified six important constituents of emu oil. The six important constituents are: linolenic acid, linoleic acid, palmitoleic acid, oleic acid, stearic acid, and palmitic acid. The present disclosure identifies specific combinations of plant derived oils and fatty acids isolates that mimic or are very similar to emu oil.

These combinations may include plant oils that are high in palmitoleic acid, in combination with plant oils that are high in oleic acid, in combination with plant oils that are high in linoleic acid, in combination with plant oils that are high in linolenic acid, in combination with plant oils that are high in stearic acid, in combination with plant oils that are high in palmitic oil. The formulas contained are percentage ratios of fatty acids.

In one embodiment, plant oils that are high in palmitoleic acid include sea buckthorn berry seed oil (genus Hippophae) and/or macadamia nut seed oil (genus Macadamia) and/or avocado seed oil (genus Persea).

In another embodiment, plant oils that are high in oleic acid include macadamia nut seed oil (genus Macadamia) and/or moringa oil (genus Moringa) and/or olive oil (genus Olea), and/or avocado seed oil (genus Persea).

In another embodiment, plant oils that are high in linoleic acid include cotton seed oil (genus Gossypium) and/or safflower oil (genus Carthamus), and/or argan oil (genus Argania), and/or passion fruit seed oil (genus Passifloraceae), and/or hemp seed oil (genus Cannabis), and/or avocado seed oil (genus Persea), and/or flax seed oil (genus Linum), and or palm kernel oil (genus Elaeis).

In another embodiment, plant oils that are high in linolenic acid include flaxseed oil (genus Linum) and/or hemp seed oil (genus Cannabis), and/or buckthorn berry seed oil (genus Hippophae).

In another embodiment, plant oils that are high in stearic acid include cocoa butter (genus Theobroma), and/or shea nut oil (genus Vitellaria).

In another embodiment, plant oils that are high in palmitic oil include palm kernel oil (genus Elaeis) and/or cocoa butter (genus Theobroma), and/or avocado seed oil (genus Persea), and/or sea buckthorn berry seed oil (genus Hippophae).

To obtain the closest possible ratios of the six major components of emu oil, certain plant-derived fatty acid isolates may be incorporated into formulas. For example, plant-derived oleic acid may be used to create optimal fatty acid ratios that mimic emu oil. It is cost prohibitive or difficult to manufacture or to create a plant-derived emu-like oil with virtually identical ratios of the 6 main fatty acids of emu oil. Therefore, the utilization of 2-6 plant derived oils in combination with 0-4 plant derived fatty acid isolates is considered unique and novel in creating a near identical fatty acid profile to that of animal-based emu oil. This would limit the number of oils and henceforth, the cost in developing an emu equivalent blend. However, the consumer marketplace may demand blends that are plant oils and not of plant oil isolates. This is because the process involved in creating the individual fatty acid isolates may be considered undesirable.

### DETAILED DESCRIPTION OF THE INVENTION

In one embodiment, the present disclosure provides a consistent, bio-similar non-animal emu oil composition.

In one embodiment, the present disclosure includes a combination of plant and other non-animal based oils to provide an oil that is effective for the same uses that emu oil is effective for.

At the outset, it was important to identify the most important fatty acid constituents of emu oil. The present disclosure has identified these components, which are disclosed below in Table 1.

TABLE 1

| | | | Chemical |
|---|---|---|---|
| Common Name | IUPAC Name | Other Name | Abbreviation |
| Palmitic | Hexadecanoic acid | | C16:0 |
| Palmitoleic | 9-hexadecenoic acid | omega-7 | C16:1n-7 |
| Stearic | Octadecanoic acid | | C18:0 |
| Oleic | Octadecenoic acid | omega-9 | C18:1n-9 |
| Linoleic | cis, cis-9,12- | omega-6 | C18:2n-6 |
| | Octadecadienoic acid | | |
| Linolenic | (9Z,12Z,15Z)-octadeca-9, | omega-3 | C18:3n-3 |
| | 12,15-trienoic acid | | |

As discussed, emu oil has differing compositions depending on a many factors. The present disclosure identifies an average for each of the most important fatty acids. See Table 2 below.

TABLE 2

| Fatty Acid Averages | Percentage by Weight |
|---|---|
| Palmitic | 24.0 |
| Palmitoleic | 4.3 |
| Stearic | 8.5 |
| Oleic | 49.1 |
| Linoleic | 9.5 |
| Linolenic | 1.1 |
| other | 3.5 |

Table 3 below depicts the fatty acid compositions of several different plant oils, fatty acids, and fatty acid isolates. In some examples, due to other minor constituents and rounding, the compositions do not all add up to 100%

TABLE 3

| Common Name | Palmitic | Palmitoleic | Stearic | Oleic | Linoleic | Linolenic | Total %* |
|---|---|---|---|---|---|---|---|
| Emu Oil | 22 | 4 | 9 | 50 | 10 | 1 | 96 |
| Olive Oil | 10.4 | 0.7 | 3.1 | 73.9 | 8.4 | 0.7 | 97.2 |
| Macadamia Nut | 8 | 20 | 4 | 62 | 2 | 0 | 96 |
| Oil | | | | | | | |
| Sea Buckthorn | 29 | 36 | 1.5 | 26 | 2.5 | 1.5 | 96.5 |
| Berry | | | | | | | |
| Flax Seed Oil | 3 | 0.1 | 7 | 21 | 16 | 53 | 100.1 |
| Cotton Seed Oil | 22 | 1 | 3 | 19 | 54 | 1 | 100.0 |
| Safflower Oil | 7 | 0.1 | 2 | 13 | 78 | 0 | 100.1 |
| Cocoa Butter | 25 | 0.3 | 38 | 32 | 3 | 0 | 98.3 |
| Moringa Oil | 5.8 | 1.4 | 6.2 | 70.2 | 0.4 | 0 | 84 |
| Oleic Acid | 0 | 0 | 0 | 100 | 0 | 0 | 100 |
| Palmitic Acid | 100 | 0 | 0 | 0 | 0 | 0 | 100 |
| Stearic Acid | 0 | 0 | 100 | 0 | 0 | 0 | 100 |
| Palm Kernel Oil | 44.6 | 0.3 | 4.4 | 40.5 | 10.1 | 0.2 | 100.1 |
| Shea Nut Oil | 4.1 | 0 | 45.8 | 42.5 | 6.1 | 0 | 98.5 |
| Hemp Oil | 5.9 | 0.1 | 2.6 | 12.2 | 55.9 | 20.4 | 97.1 |
| Argon Oil | 13 | 0.1 | 5.8 | 46 | 33 | 0.2 | 98.1 |
| Passion Fruit | 10.3 | 0.3 | 3 | 14.7 | 70.8 | 0.3 | 99.4 |
| Seed Oil | | | | | | | |
| Avocado Seed | 28.2 | 5.7 | 0.7 | 50.95 | 13.9 | 0.6 | 100.05 |
| Oil | | | | | | | |

In a preferred embodiment, the consistent, bio-similar emu oil equivalent has a target fatty acid composition of 24% palmitic acid, 4.3% palmitoleic acid, 8.5% of stearic acid, 49.1% oleic acid, 9.5 linoleic acid, and 1.1% of linolenic acid.

Table 4 depicts an analysis of the fatty acid composition of emu oils that were deemed to be highly effective for various medical and therapeutic uses.

TABLE 4

| Fatty Acid | | | | | | | | | | Emu |
|---|---|---|---|---|---|---|---|---|---|---|
| Composition | % | % | % | % | % | % | % | % | % | Target |
| Palmitic | 25.2 | 25.2 | 25 | 25.2 | 25.2 | 25.2 | 25.2 | 25.2 | 25.1 | 24 |
| Palmitoleic | 4.3 | 4.3 | 4.3 | 4.3 | 4.3 | 4.3 | 4.3 | 4.3 | 4.4 | 4.3 |
| Stearic | 8.5 | 8.8 | 8.5 | 8.5 | 8.5 | 8.5 | 8.5 | 8.5 | 8.6 | 8.5 |
| Oleic | 49.1 | 50.9 | 49.1 | 49.1 | 49.1 | 49.1 | 49.1 | 49.1 | 50.1 | 49.1 |
| Linoleic | 9.5 | 9.7 | 9.5 | 9.5 | 9.6 | 9.5 | 9.5 | 9.5 | 9.5 | 9.5 |
| Linolenic | 1.1 | 1.1 | 1.1 | 1.1 | 1.1 | 1.1 | 1.1 | 1.1 | 1.1 | 1.1 |
| Total % | 97.7 | 100 | 97.5 | 97.7 | 97.8 | 97.7 | 97.7 | 97.7 | 98.9 | 96.5 |

Table 5 depicts examples of oil compositions that are consistent and bio-similar to emu oil. The oil compositions depict plant oils and fatty acid isolates on a weight percentage basis.

TABLE 5

| Oil/Fatty | | | | | | | | | | |
|---|---|---|---|---|---|---|---|---|---|---|
| Acid | 1 | 2 | 3 | 4 | 5 | 6 | 7 | 8 | 9 | 10 |
| Olive | | 33 | | | | 23 | | 40 | 17 | |
| Macadamia | | | | | 21 | 21 | | | 20 | |
| Sea Buckthorn | 12 | 11 | 9 | 10 | | | 10 | 11 | | 2 |
| Berry | | | | | | | | | | |
| Flax Seed | 2 | | 2 | 2 | | 2 | 2 | 1 | 2 | 2 |
| Cotton Seed | 17 | | | | | | | | | |
| Safflower | | | 6 | 6 | | 3 | 6 | 3 | 3 | |
| Cocoa Butter | | | | 11 | | 1 | 11 | 15 | | |
| Moringa | | | 41 | 39 | | | 37 | | | |
| Palm Kernel | | 40 | 42 | 35 | 50 | 46 | 38 | 29 | 48 | |
| Oil | | | | | | | | | | |
| Shea Nut Oil | | 12 | | | 10 | | | | 11 | |
| Hemp Oil | | 3 | | | 5 | | | | | |
| Argan Oil | | | | | 2 | | | | | |
| Passion Fruit | | | | | | | | | | |
| Seed Oil | | | | | | | | | | |
| Avocado | | | | | | | | | | 66 |
| Oleic Acid | 44 | | | | 10 | | | | | 17 |
| Isolate | | | | | | | | | | |
| Palmitic Acid | 18 | | | | | | | | | 6 |
| Isolate | | | | | | | | | | |
| Stearic Acid | 8 | | 3 | | | 4 | | | | 8 |
| Isolate | | | | | | | | | | |
| Total % | 101 | 99 | 103 | 103 | 98 | 100 | 104 | 99 | 101 | 101 |

As an illustrative example of the compositions disclosed in Table 5, composition 1 of Table 5 is comprised of 12 wt % sea buckthorn berry oil, 2 wt % of flax seed oil, 17 wt % of cotton seed oil, 44 wt % of oleic acid isolate, 18 wt % of palmitic acid isolate, and 8 wt % of stearic acid isolate. Each composition of Table 5 could be elaborated similarly.

Table 6 depicts different plant oil sources for each of the six most important fatty acid constituents of emu oil. In some cases, the fatty acid isolate is used as the source of that fatty acid constituent. For each plant oil source or fatty acid isolate, the table indicates how much of that major constituent is present on a weight percentage basis.

The highlighted sections of the table point out where the plant oil source or fatty acid isolate has a higher weight percentage of that major fatty acid constituent. This information was used to determine the upper limit for the weight percentage of the plant oil or fatty acid isolate in each of the disclosed compositions. Finally, the table depicts the high end weight percentage of that plant oil or fatty acid isolate for the oil compositions of the present disclosure. As can be imagined, a higher weight percentage of the maximum plant oil or fatty acid isolate weight percentage listed would undesirably create an oil composition having a higher percentage of that fatty acid constituent than what is present in emu oil.

In particular, the present disclosure has identified six important components of emu oil. The six important components are: linolenic acid, linoleic acid, palmitoleic acid, oleic acid, stearic acid, and palmitic acid. The present disclosure identifies specific combinations of plant derived oils and fatty acids isolates that mimic or are very similar to emu oil.

These combinations may include plant oils that are high in palmitoleic acid, in combination with plants oils that are high in oleic acid, in combination with plant oils that are high in linoleic acid, in combination with plant oils that are high in linolenic acid, in combination with plant oils that are high in stearic acid, in combination with plant oils that are high in palmitic oil. The formulas contained are percentage ratios of fatty acids.

In one embodiment, plant oils that are high in palmitoleic acid include sea buckthorn berry seed oil (genus Hippophae) and/or macadamia nut seed oil (genus Macadamia) and/or avocado seed oil (genus Persea).

In another embodiment, plant oils that are high in oleic acid include macadamia nut seed oil (genus Macadamia) and/or moringa oil (genus Moringa) and/or olive oil (genus Olea), and/or avocado seed oil (genus Persea).

In another embodiment, plant oils that are high in linoleic acid include cotton seed oil (genus Gossypium) and/or safflower oil (genus Carthamus), and/or argan oil (genus Argania), and/or passion fruit seed oil (genus Passifloraceae), and or hemp seed oil (genus Cannabis), and/or avocado seed oil (genus Persea), and/or flax seed oil (genus Linum), and or palm kernel oil (genus Elaeis).

In another embodiment, plant oils that are high in linolenic acid include flaxseed oil (genus Linum) and/or hemp seed oil (genus Cannabis), and/or buckthorn berry seed oil (genus Hippophae).

In another embodiment, plant oils that are high in stearic acid include cocoa butter (genus Theobroma), and/or shea nut oil (genus Vitellaria).

In another embodiment, plant oils that are high in palmitic oil include palm kernel oil (genus Elaeis) and/or cocoa butter (genus Theobroma), and/or avocado seed oil (genus Persea), and/or sea buckthorn berry seed oil (genus Hippophae).

To obtain the closest possible ratios of the six major components of emu oil, certain plant derived fatty acid isolates may be incorporated into formulas. For example, plant derived oleic acid may be used to create optimal fatty acid ratios that mimic emu oil. It is difficult, cost prohibitive, or difficult to manufacture or to create a plant-derived emu-like oil with virtually identical ratios of the 6 main fatty acids of emu oil. Therefore, the utilization of 2-6 plant derived oils in combination with 0-4 plant derived fatty acid isolates is considered unique and novel in creating a near identical fatty acid profile to that of animal-based emu oil. This would limit the number of oils and henceforth the cost in developing an emu equivalent blend. However, the consumer marketplace may demand blends that are plant oils and not of plant oil isolates. This is because the process involved in creating the individual fatty acid isolates may be considered undesirable.

In one embodiment, a method of making a consistent bio-similar emu oil composition includes the steps of combining the oils and fatty acids at a temperature that is capable of melting the oils and fatty acids. It should be noted that emu oil has a melting point close to that of room temperature and therefore may exist as a liquid, solid, or combination of liquid and solid at room temperature. The combining step may utilize a rotary mixer set to standard prop agitation.

In another embodiment, an anti-oxidant is added to the oil composition. ‘Good’ oils have unsaturated bonds, thereby making them susceptible to oxidation and breakdown. This breakdown may produce rancidity, color change, and undesirable tastes and odors. This breakdown may also make the oils sub-optimal in cosmetic or medical performance. One solution to this is to refrigerate the oils though, in many cases, this is not practical. Thus an anti-oxidant can aid in the integrity of the unsaturated bonds, thereby adding to the shelf life of the oil composition. In one embodiment, natural antioxidants may be used to add shelf life. In a preferred embodiment, mixed tocopherols containing alpha, beta, delta, and gamma tocopherols may be used. In other embodiments, other antioxidants may be used such as butylated hydroxytoluene commonly known as BHT and butylated hydroxyanisole commonly known as BHA. In further embodiments, many other natural and synthetic antioxidants may be used that will also aid in the stability of the oil blend. In another embodiment, the antioxidant may be vitamin c, ascorbic acid, ascorbic palmitate, rosemary oil, or combinations thereof.

In other embodiments, oil composition may include ascorbic acid, ascorbic palmitate, other ascorbic acid derivatives, rosemary oil, alpha tocopherol, beta tocopherol, delta tocopherol, gamma tocopherol, butylated hydroxytoluene, butylated hydroxyanisole, and combinations thereof.

### EXAMPLES

### Example 1

It is necessary to ensure that the bio-similar emu oil composition does not cause allergic reactions. Thus, a skin prick test was conducted to test the allergen potential of the oil composition disclosed.

To further show the comparability of bio-similar emu oil to emu oil, a skin prick allergy test was performed. Plant-based products may cause allergic reactions. However, we demonstrated that the bio-similar emu oil does not produce these effects.

To test the allergen potential of bio-similar emu oil and emu oil, drops of the following substances were placed on the arm through which a single lancet was administered, between the elbow and wrist of each subject. After 15 minutes, the results were recorded as this is the proper amount of time for the visual inflammatory effects of the histamine to present on the skin. The subjects were screened to ensure they did not have any creams or lotions on the skin at the time and had not taken any antihistamine drugs for 72 hours before the test was performed. A drop of the possible allergen solution was placed by each code written on the arm of the subject:

Positive control: drop of 1 mg/mL histamine

Negative control: drop of saline

Drop of bio-similar emu oil Version 1 (Semi-Solid)

Drop of bio-similar emu oil Version 2

Drop of bio-similar emu oil Version 3

Drop of bio-similar emu oil Version 4

Drop of emu oil

The experimental treatments resulted in a small pink dot where the lancet touched the skin through the treatment in all cases of all participants of which were 8. The emu oil and the bio-similar emu oil versions 1-4 had an identical effect to each other, to the saline, the negative control. Histamine, the positive control, caused a weal to be formed on the skin of all participants. This is the expected result of treating with an allergen causing a typical inflammatory reaction.

This data shows that bio-similar emu oil Versions 1-4 are comparable to emu oil as well as saline in the respect that they do not cause an allergic or inflammatory reaction in non-sensitive subjects. A sensitive subject would be one that exhibits inflammation after treatment with the saline. This test demonstrates that the bio-similar emu oil compositions is equivalent and is a proper analogue of emu oil with respect to potential allergic reactions and inflammatory effects.

It will be appreciated by those skilled in the art that while the oil composition has been described in detail herein, the invention is not necessarily so limited and other examples, embodiments, uses, modifications, and departures from the embodiments, examples, uses, and modifications may be made without departing from the oil composition and all such embodiments are intended to be within the scope and spirit of the appended claims.

What is claimed is:

1. An oil composition comprising:
a source of palmitoleic acid, wherein the source of palmitoleic acid is selected from the group consisting of olive oil, macadamia nut oil, sea buckthorn berry oil, flax seed oil, cotton seed oil, safflower oil, cocoa butter, moringa oil, palm kernel oil, hemp oil, argan oil, passionfruit oil, avocado oil, and combinations thereof;
a source of oleic acid, wherein the source of oleic acid is selected from the group consisting of olive oil, macadamia nut oil, sea buckthorn berry oil, flax seed oil, cotton seed oil, safflower oil, cocoa butter, moringa oil, palm kernel oil, shea nut oil, hemp oil, argan oil, passionfruit oil, avocado oil, and combinations thereof;
a source of linoleic acid, wherein the source of linoleic acid is selected from the group consisting of olive oil, macadamia nut oil, sea buckthorn berry oil, flax seed oil, cotton seed oil, safflower oil, cocoa butter, moringa oil, palm kernel oil, shea nut oil, hemp oil, argan oil, passionfruit oil, avocado oil, and combinations thereof;
a source of linolenic acid wherein the source of linolenic acid is selected from the group consisting of olive oil, sea buckthorn berry oil, flax seed oil, cotton seed oil, palm kernel oil, hemp oil, argan oil, passionfruit oil, avocado oil, and combinations thereof;
a source of stearic acid wherein the source of stearic acid is selected from the group consisting of olive oil, macadamia nut oil, sea buckthorn berry oil, flax seed oil, cotton seed oil, safflower oil, cocoa butter, moringa oil, palm kernel oil, shea nut oil, hemp oil, argan oil, passionfruit oil, avocado oil, and combinations thereof; and
a source of palmitic acid wherein the source of palmitic acid is selected from the group consisting of olive oil, macadamia nut oil, sea buckthorn berry oil, flax seed oil, cotton seed oil, safflower oil, cocoa butter, moringa oil, palm kernel oil, shea nut oil, hemp oil, argan oil, passionfruit oil, avocado oil, and combinations thereof.

2. The oil composition of claim 1 further comprising an antioxidant selected from the group consisting of ascorbic acid, ascorbic palmitate, other ascorbic acid derivatives, rosemary oil, alpha tocopherol, beta tocopherol, delta tocopherol, gamma tocopherol, butylated hydroxytoluene, butylated hydroxyanisole, and combinations thereof.

3. The oil composition of claim 2, wherein olive oil is present in an amount up to 66 wt %.

4. The oil composition of claim 2, wherein macadamia nut oil is present in an amount up to 22 wt %.

5. The oil composition of claim 2, wherein sea buckthorn berry oil is present in an amount up to 12 wt %.

6. The oil composition of claim 2, wherein flax seed oil is present in an amount up to 2 wt %.

7. The oil composition of claim 2, wherein cotton seed oil is present in an amount up to 18 wt %.

8. The oil composition of claim 2, wherein safflower oil is present in an amount up to 12 wt %.

9. The oil composition of claim 2, wherein cocoa butter is present in an amount up to 5 wt %.

10. The oil composition of claim 2, wherein moringa oil is present in an amount up to 70 wt %.

11. The oil composition of claim 2, wherein palm kernel oil is present in an amount up to 54 wt %.

12. The oil composition of claim 2, wherein shea nut oil is present in an amount up to 19 wt %.

13. The oil composition of claim 2, wherein hemp oil is present in an amount up to 0.1 to 5 wt %.

14. The oil composition of claim 2, wherein argan oil is present in an amount up to 5 wt %.

15. The oil composition of claim 2, wherein passionfruit oil is present in an amount up to 14 wt %.

16. The oil composition of claim 2, wherein avocado oil is present in an amount up to 69 wt %.

17. An oil composition comprising:
a source of palmitoleic acid, wherein the source of palmitoleic acid is selected from the group consisting of olive oil, macadamia nut oil, sea buckthorn berry oil, flax seed oil, cotton seed oil, safflower oil, cocoa butter, moringa oil, palm kernel oil, hemp oil, argan oil, passionfruit oil, avocado oil, and combinations thereof;
a source of oleic acid, wherein the source of oleic acid is selected from the group consisting of olive oil, macadamia nut oil, sea buckthorn berry oil, flax seed oil, cotton seed oil, safflower oil, cocoa butter, moringa oil, palm kernel oil, shea nut oil, hemp oil, argan oil, passionfruit oil, avocado oil, oleic acid isolates, and combinations thereof;
a source of linoleic acid, wherein the source of linoleic acid is selected from the group consisting of olive oil, macadamia nut oil, sea buckthorn berry oil, flax seed oil, cotton seed oil, safflower oil, cocoa butter, moringa oil, palm kernel oil, shea nut oil, hemp oil, argan oil, passionfruit oil, avocado oil, and combinations thereof;
a source of linolenic acid wherein the source of linolenic acid is selected from the group consisting of olive oil, sea buckthorn berry oil, flax seed oil, cotton seed oil, palm kernel oil, hemp oil, argan oil, passionfruit oil, avocado oil, and combinations thereof;
a source of stearic acid wherein the source of stearic acid is selected from the group consisting of olive oil, macadamia nut oil, sea buckthorn berry oil, flax seed oil, cotton seed oil, safflower oil, cocoa butter, moringa oil, palm kernel oil, shea nut oil, hemp oil, argan oil, passionfruit oil, avocado oil, stearic acid isolates, and combinations thereof; and
a source of palmitic acid wherein the source of palmitic acid is selected from the group consisting of olive oil, macadamia nut oil, sea buckthorn berry oil, flax seed oil, cotton seed oil, safflower oil, cocoa butter, moringa oil, palm kernel oil, shea nut oil, hemp oil, argan oil, passionfruit oil, avocado oil, palmitic acid isolates, and combinations thereof.

18. The oil composition of claim 17, wherein the oleic acid isolate is present in an amount up to 50 wt % and wherein the oil composition further comprises an antioxidant selected from the group consisting of ascorbic acid, ascorbic palmitate, other ascorbic acid derivatives, rosemary oil, alpha tocopherol, beta tocopherol, delta tocopherol, gamma tocopherol, butylated hydroxytoluene, butylated hydroxyanisole, and combinations thereof.

19. The oil composition of claim 17, wherein the stearic acid isolate is present in an amount up to 9 wt % and wherein the oil composition further comprises an antioxidant selected from the group consisting of ascorbic acid, ascorbic palmitate, other ascorbic acid derivatives, rosemary oil, alpha tocopherol, beta tocopherol, delta tocopherol, gamma tocopherol, butylated hydroxytoluene, butylated hydroxyanisole, and combinations thereof.

20. The oil composition of claim 17, wherein the palmitic acid isolate is present in an amount up to 24 wt % and wherein the oil composition further comprises an antioxidant selected from the group consisting of ascorbic acid, ascorbic palmitate, other ascorbic acid derivatives, rosemary oil, alpha tocopherol, beta tocopherol, delta tocopherol, gamma tocopherol, butylated hydroxytoluene, butylated hydroxyanisole, and combinations thereof.
